(19) United States
(12) Patent Application Publication
(10) Pub. No.: US 2018/0035236 A1
(43) Pub. Date: Feb. 1, 2018

(54) Audio System with Binaural Elements and Method of Use with Perspective Switching

(71) Applicants: Leonardo Basterra, Katy, TX (US); Maria Isabel Tapias, Katy, TX (US)

(72) Inventors: Leonardo Basterra, Katy, TX (US); Maria Isabel Tapias, Katy, TX (US)

(21) Appl. No.: 15/658,909

(22) Filed: Jul. 25, 2017

Related U.S. Application Data
(60) Provisional application No. 62/367,873, filed on Jul. 28, 2016.
(60) Provisional application No. 62/367,865, filed on Jul. 28, 2016.

(51) Int. Cl. H04S 7/00; A61M 21/02; H04S 5/00; G06F 3/16; G06F 3/0482; G06F 3/0481

(52) U.S. Cl. CPC H04S 7/304; G06F 3/0482; G06F 3/04817; H04S 5/00; G06F 3/167; A61M 21/02; H04S 2400/01; H04S 2400/11; H04W 4/008

(57) ABSTRACT

An audio system with binaural elements with a computer processor connected to data storage. The audio system is capable of perspective switching between different binaural elements present in an audio recording.

### CROSS-REFERENCE TO RELATED APPLICATIONS

This application claims priority to U.S. provisional patent application 62/367,873 filed on Jul. 28, 2016 and 62/367,865 filed on Jul. 28, 2016 both of which are incorporated by reference herein as if set forth in their entirety.

### STATEMENT REGARDING FEDERALLY SPONSORED RESEARCH

Not applicable.

### BACKGROUND

In some embodiments, the present embodiments generally relate to a system for playback of an audio recording with binaural audio recording.

In some embodiments, the present embodiments generally relate to an audio recording that has three-dimensional (3D) audio or has a sound environment that allows the listener to determine, and select, the audio source location and perspective. In several embodiments, the sounds around them are created by using perspective switching during playback, enabling the listener to become more aware of their surroundings.

In some embodiments, the present embodiments generally relate to a system to allow the visually impaired to hear materials to identify proximity, by using one or more of a variety of preset sound metrics to identify their location, each sound metric providing stimuli within a frequency range for perspective.

In some embodiments, the present embodiments generally relate to a system for individuals with learning disabilities to process content, by using one or more of a variety of preset sound tools to process educational materials, each sound metric providing stimuli within a frequency range for mental focus.

In some embodiments, the present embodiments generally relate to a system to allow technical professionals to hear materials to identify hazards, by using one or more of a variety of preset sound metrics to identify right from wrong, each sound metric providing stimuli within a frequency range for evaluation.

The present invention is distinguished from the following prior art pieces:

“Binaural Audio Guide” EP 2,214,425 A1 issued on Jan. 28, 2009 is an invention that is preferably for use in museums, provides users with information about the objects in such a manner that the information provided seems to come from the specific objects. One difference from the present invention is that the object generating sound moves around the listener in the present invention versus having a still object generate sound from one display location.

“Binaural Audio Wave ‘Pickup for Musical Instruments” U.S. Pat. No. 2,938,419 from May 31, 1960 is designed to “produce a minimum amount of distortion” Its function is to pick up and collect binaural audio waves from an accordion. The invention of '419 is irrelevant to the present invention.

“Apparatus for Creating 3D Audio Abstract Imaging Over Headphones Using Binaural Synthesis” U.S. Pat. No. 5,809,149 from Sep. 25, 1996 is an invention for audio signals for playback over headphones in which an apparent sound location can be smoothly panned over a number of locations without requiring an unduly complex circuit. One difference from the present invention is that the present invention uses several audio stimuli, like original recordings through 3D microphones, recorded character voices and combines all those elements using a combination of hardware and software to generate an optimal 3D audio effect.

“Dynamic Decoding of Binaural Audio Signals” U.S. Pat. No. 7,876,904 B2 from Jan. 25, 2011 is a binaural decoder system to allow speakers to deliver 3D-like audio through surround sound. Binaural Cue Coding (BCC) is a highly developed para metric spatial audio coding method designed for multi-channel loudspeaker systems. The focus of the present invention is on the production and post-production components (recordings, software, mixing) to create a 3D audio experience with movement and must be deliverable through earbuds or headphones.

“Generating 3d Audio Using a Regularized Hrtf/Hrir Filter” U.S. Pat. No. 7,912,225 from Mar. 22, 2011 is an improved smoothness in the HRTF filter to allow the perception by the listener of a smoothly moving sound rendering free of annoying sound distortion. This patent is generally not relevant to the present invention.

“Controlling the Decoding of Binaural Audio Signals” U.S. Pat. No. 8,081,762 from Dec. 20, 2011 is a Spatial audio coding, and more particularly to control the decoding of binaural audio signals; with a focus on delivering through speakers. This invention is not directly relevant to the present invention.

“3D Audio Renderer” U.S. Pat. No. 8,488,796 from Jul. 16, 2013 is an invention that uses signal processing techniques. More particularly, the present invention relates to methods for processing audio signals. This patent is not directly relevant to the present invention.

“Binaural Audio Signal-Based Applications” U.S. Pat. No. 8,552,860 from Oct. 8, 2013 is an invention that uses applications involving binaural sounds, and more particularly to, among other things, detecting binaural signals, blocking binaural signals and customizing binaural signals for a specific individual—with the goal to have cognitive impact. The invention of this patent includes English/Spanish translator to leverage relevant sides of the brain for the desired effect. The present inventive device focus is on the content and the production, post production to achieve the same cognitive effects vs. manipulating/controlling the binaural signal.

“Method and Apparatus for Generating 3D Audio Positioning Using Dynamically Optimized Audio 3D Space Perception Cues” U.S. Pat. No. 8,755,432 from Jun. 17, 2014 is an invention for the preparation of digital 3D movies for release in one format it does not include an efficient conversion of the audio component that insures the presence or preservation of the 3D space perception audio cues. This invention pertains to a method and apparatus of enhancing a 3D movie by using 3D space information associated with at least some of the characters/objects that are either part of the scene or off scene during the preparation of a digital 3D movie. Its audio component may not include clear 3D space perception cues, either because these cues have been stripped away or because they were missing. This patent is not directly relevant to the present invention.

“Method and System for Up Mixing Audio to Generate 3D Audio” U.S. Pat. No. 9,094,771 from Jul. 28, 2015 is a coding method which extracts parts of an existing 2D audio program to generate an upmixed 3D audio program which when rendered by speakers is perceived as having depth effects. The goal is to generate 3D audio through speakers at different distances from the listener to aid visual reference and/or be stimulated by “stereoscopic 3D video programs.” The invention of this patent is not directly related to the present inventive device.

“System and Tools for Enhanced 3D Audio Authoring and Rendering” U.S. Pat. No. 9,204,236 from Dec. 1, 2015 is an invention that relates to authoring and rendering of audio reproduction data for reproduction environments such as cinema sound. Refers to tools that create metadata for audio objects, specifically speaker zones and layout. The invention of this patent does not mention the use of perspective switching.

“System and Tools for Enhanced 3D Audio Authoring and Rendering” U.S. Pat. No. 9,549,275 from Jan. 17, 2017 is essentially the same as U.S. Pat. No. 9,204,236.

“System and Method for Producing Had Externalized 3D Audio Through Headphones” U.S. Pat. No. 9,560,464 from Jan. 31, 2017 essentially relates to 3D technology inserted into headphones, using filters, crosstalk cancellation. Input is any kind of audio and pretends to translate into a 3D representation. The present invention has input that is audio that has been created using sources and tools that generate binaural and 3D environments. We do not use any specific technology for the headphones. No mention of perspective switching.

“Implementation Method Of 3D Audio” Pat. App. US 2003/0202665 published Oct. 30, 2003 uses head related transfer function to synthesize binaural sound from a monoaural source. This again, is applying an audio algorithm to regular sound to make it sound like binaural. This invention could be used inside headphones to convert regular monoaural sound into binaural sound. The present invention uses headphones and perspective switching.

“Decoding of Binaural Audio Signals” Pat. App. US 2007/0160218 published Jul. 12, 2007 is an invention that applies a predetermined set of head-related transfer function filters to a plurality of audio channels. Refers particularly to decoding of binaural audio signals to simulate in many audio sources. The key difference between '218 and the present invention is that this invention has nothing to do with headphone delivery. '218 appears to refer to how to simulate 3D sound using multiple audio channels or sources. No perspective switching.

“Apparatus and Method for Processing 3D Audio Signal Based on Hrtf, And Highly Realistic Multimedia Playing System Using the Same” Pat. App. US 2011/0150098 from Jun. 23, 2011 is hardware that decodes audio data to translate it into 3D data. The key between the present invention and difference that of '098 is that '098 refers to a binaural microphone but not to the content per se. '098 neither mentions, headphone delivery nor perspective switching.

“Binaural Audio Systems and Methods” Pat. App. US 2015/0296288 from Oct. 15, 2015 is related to audio information that can be stored in a mobile device to sync up with a speaker-like device. Refers to the ability to experience movies in different languages via headphones. The above patent application refers to the storage of binaural audio, not the creation of binaural/3D content. The above patent application has not much to do with the audio per se, but rather the delivery of specific audio. The above patent application does not refer to perspective switching.

“Binaural Audio Processing” Pat. App. US 2015/0350801 refers to an audio renderer and processor of head related binaural transfer function data. This application has no mention of headphone delivery or perspective switching.

“Passive Audio Headset Compatible with Binaural Recording and Off-Headset Noise Cancellation” Pat. App. US 2016/0373861 refers to the headset, and specific functions for binaural recording and noise cancellation. This application does not relate to the creation of binaural content and mixing. This application does not refer to scripted stories and the delivery mechanisms. This application does not refer to perspective switching.

“Binaural Audio Systems and Methods” Pat. App. US 2017/0034638 is substantially the same as US 2015/0296288 A1.

### SUMMARY

Several embodiments relate to a system and method of use of a binaural audio recording, which can have a computing system with a computer processor connected to the data storage, wearable head gear, stereo audio output, ear buds, Bluetooth device, and a listener personal computing device.

Several embodiments can give the listener the ability to adjust the type of loudness of sources playing back. Several embodiments can provide a series of neurological therapy sounds that comfort the listener.

Several embodiments of the present invention can assist people working in a manufacturing or production environment with machinery sounds by training those workers to be perceptive of the sounds of the machines around them and the associated meaning of those sounds. In embodiments, the listener may be able to click on certain sounds to connect to instructions stored in the data storage on how to repair the machinery, which allows the listener to become familiar with sounds related to dangerous conditions, such as an explosion or fire.

In several embodiments, the data storage can store an audio recording with a binaural audio recording and have computer instructions to enable the listener to select one of the audio recordings from the menu of audio recordings, to view a menu of principal characters that experience the story in the selected audio recording, and to select a principal character to initiate the story of the selected audio recording, to play, stop, pause, reverse, forward, fast reverse, fast forward, mark stop for resume play at another time, and to change primary characters at any point in the story.

In several embodiments, the present invention is a system for playback of an audio recording with a binaural audio recording, comprising: an audio recording comprising: a binaural audio recording; a menu list with items; a menu selection button to select items from the menu listing name; a data storage configured to store at least one audio recording having the binaural audio recording and to present a series of audio recording choices to a listener, the binaural audio recording comprising a member, or combination of members, from the group consisting of: holophonic sound effects; a dialog; music; narration; and/or script; a computing system comprising: a computer processor connected to the data storage, the computer processor configured to receive and playback the audio recording as an audio output signal; wearable headgear in communication with the computer processor configured to receive and play the audio output signal through Bluetooth transmission, and/or a first output and a second output; a listener personal computing device connected to the computer processor; said data storage further comprising: computer instructions for instructing the computer processor to present a series of audio recording choices to the listener and enable the listener to select one of the audio recordings from the series of audio recording choices using the listener personal computing device, each audio recording having the binaural audio recording; a perspective switching button viewable on the display and connected to computer instructions configured to instruct the personal computing device processor and/or computer processor to present non-selected individual audio sources to the listener for selection at any time during the playback; a plurality of navigation buttons viewable, physically available, or audio activated on the display and connected to computer instructions to instruct the personal computing device processor and/or computer processor to perform a selected navigational task when a selected navigational button is activated by the listener enabling the listener to move through the binaural audio recording.

In several embodiments, the data storage comprises computer instructions configured to instruct the personal computing device processor and/or computer processor to download or stream a voice activation and voice control to the personal computing device data storage and provide voice control of any binaural audio recording enabling a listener to control the playback, and wherein the listener's personal computing device comprises a microphone for detecting audio commands from a listener and triggering at least one of: the recording icon, the binaural audio recording, the perspective switching button and the plurality of navigation buttons. In several embodiments, the listener's personal computing device is a smart phone, mobile computing device, laptop, tablet, personal computer, headphones, earbuds or similar computing device with memory. In several embodiments, the wearable headgear are headphones, earbuds, blue tooth accessories, or any device having an audio transmitter with a direct connection to the listener's ears.

In several embodiments, the computer processor and data storage comprise at least one of: a smart phone, mobile computing device, laptop, tablet, personal computer, headphones, earbuds, or computing device with memory. In several embodiments, the computer instructions for instructing the computer processor to connect the computer processor to a network to download or stream additional audio recordings from an online store. In several embodiments, the network is a cellular network, a global communication network, a drone, a satellite network, a local area network, a wide area network, Bluetooth transmission, or combinations thereof.

In several embodiments, the binaural audio recording allows the listener to define sound quality metrics for size, intensity, position, distance, dynamics, tempo, and type of sound. In several embodiments, the binaural audio recording further comprising at least one of: a non-holophonic sound effect and/or music. In several embodiments, the script is a music score and the individual audio source is an instrument presenting a portion of the music score or the individual audio source is an audience viewing the presentation of the music score. In several embodiments, the script is a series of machine sounds in a manufacturing or production environment.

In several embodiments, the audio recording is a series of neurological therapy sounds for at least one of: meditation, motivation, hypnosis therapy, relaxation, and improved concentration. In several embodiments, the script is a play and the individual audio source is a character in the play or the audience viewing the play. In several embodiments, the network is a cellular network, a global communication network, a drone, a satellite network, a local area network, a wide area network, Bluetooth transmission, or combinations thereof. In several embodiments, the personal computing device data storage comprises voice recognition software. In several embodiments, the binaural audio recording comprises a visual image button for an individual audio source, the visual image button linked to at least one visual image, the visual image corresponding to a portion of the playback.

In several embodiments, the creation of the binaural materials occurs in the following manner: 1) The first step is focused on content design based on human ears' spatial perspective to achieve a key objective and/or benefit. The goal for the script is to create a map of how an individual will experience different sound sources from different perspectives. In the script or sound map, the audio experience is outlined, scripted and/or designed for it to make sense from a spatial perspective; purely through the ears. So, sounds coming from the: left, right, front, back, top, bottom, and so forth, have to make sense to the human brain to convey an objective. 2) Audio engineers use the content/script design to identify what type of recordings are needed. This will include but is not limited to: 3D audio recording, mono audio recordings, binaural beats, Foley and regular sound effects. All the audio elements need to be recorded and then edited to achieve the audio experience objective. 3) Once the final edit is complete, the final mix refines the sound quality and makes emphasis where needed. This might include increasing or lowering the volume at key points within the recording; among many other final enhancements; depending on the objective. The final output is an audio experience that tricks the human brain into thinking it is experiencing something as if in the middle of the audio stimuli; when the person is physically absent.

In several embodiments, the present invention is a system for the creation of the binaural materials comprising the following steps: designing content based on human ears' spatial perspective to achieve a key objective and/or benefit; utilizing the content/script design to identify what type of recordings are needed for binaural effects in an audio mix; refining the audio mix to trick the human brain into thinking it is experiencing something as if in the middle of the audio stimuli; when the person is physically absent from said stimuli.

In several embodiments, the present invention is a system for playback of an audio recording with a binaural audio recording, comprising: an audio recording comprising: a binaural audio recording; a menu selection button or uses audio to activate to select features from a menu listing; a data storage configured to store at least one audio recording having the binaural audio recording and to present a series of audio recording choices to a listener; a computing system comprising: a computer processor connected to the data storage, the computer processor configured to receive and playback the audio recording as an audio output signal or Bluetooth; in communication with the computer processor configured to receive and play the audio output signal through a first output and a second output or Bluetooth; and a listener's personal computing device connected to the computer processor or transmitted through Bluetooth signal.

### BRIEF DESCRIPTION OF THE DRAWINGS

The detailed description will be better understood in conjunction with the accompanying drawings as follows:

FIG. 1 depicts a diagram of the system according to one or more embodiments.

FIG. 2 depicts a diagram of the wearable headgear according to one or more embodiments.

FIG. 3 depicts a diagram of the input device according to one or more embodiments.

FIGS. 4A, 4B, and 4C depicts data storage according to one or more embodiments.

FIG. 5 depicts the audio recording or software application as presented to the listener according to one or more embodiments.

FIG. 6 is a chart of an exemplary binaural audio recording as well as an exemplary display on a personal computing device with a display, according to one or more embodiments.

FIG. 7 illustrates one embodiment of a person surrounded by different potential sound directions

FIG. 8 illustrates another embodiment of one aspect of the present invention.

FIG. 9 illustrates one embodiment of an example of a music score.

FIG. 10 illustrates one embodiment of an example of machinery sounds as used in the present invention.

FIG. 11 illustrates one embodiment of an example of a sporting event sounds as used in the present invention.

FIG. 12 illustrates one embodiment of the audio recording process as used in the present invention.

FIG. 13 illustrates another embodiment of one aspect of the present invention.

FIG. 14 illustrates one embodiment of the creation of binaural materials.

The present embodiments are detailed below with reference to the listed Figures.

### DETAILED DESCRIPTION OF THE EMBODIMENTS

Before explaining the present apparatus in detail, it is to be understood that the apparatus is not limited to the particular embodiments and that it can be practiced or carried out in various ways.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless or essentially meaningless, the definition should be taken from Webster's Dictionary, 11th Edition, 2008. Definitions and/or interpretations should not be incorporated from other patent applications, patents, or publications, related or not, unless specifically stated in this specification or if the incorporation is necessary for maintaining validity. Specifically, defined terms:

The term “binaural audio recording” as used herein can refer to auditory illusion perceived when different pure-tone waves, with different frequencies, are presented to a listener dichotically, that is one through each ear, but with simultaneous stimulation of both ears with the same stimulus. The inequality of the two impressions determines the correct direction and distance of the sound. The binaural audio recording is a 3D audio recording, a holophonic recording, a holo-acoustic recording, and virtual reality sound. The term “binaural audio recording” can refer to a recoding that includes the script, but supplemental information as well, such as an interview with a conductor, the history of a musician, a physician endorsement, a voice over that is not included in the script, and bloopers.

The term “audio recording” as used herein can refer to any audio materials, including music, instructions, educational materials, books, pod casts, magazines, novels, instructional, manuals, self-development and improvement materials, reference manuals, and any other types of literature with a beginning, middle and end to each spoken section, which can be streamed from an online source or downloaded onto a data storage device.

The term “marked locations” as used herein can refer to marked sections of audio pieces.

The term “dialog” as used herein can refer to the words spoken by voice actors, including voice effects.

The term “holophonic sound effect” as used herein can refer to sound effects recorded, which take into account natural ear spacing or “head shadow” of the head and ears. Natural ear spacing can enable holophonic sound effects to cause a listener to generate interaural time differences and interaural level differences simultaneously. For example, a car motor moving from left to right while indicating (i) how high from the ground the car can be located relative to the listener, (ii) how far the car is from the listener, and (iii) a location of the car relative to the listener, through the binaural sound recording there can be a holophonic sound effect.

The terms “menu listing names” as used herein can refer to a display featuring a title of the audio recording, a call to action, such as “click to select character” a plurality of actuation buttons or icons enabling a listener to click or select a button, an image of a principal character, or audio passage associated with the actuation button, and an identifier that is associated with the principal character, such as a name or a description.

The term “menu of audio recordings” as used herein can refer to a display screen presenting a plurality of visual icons to a listener.

The term “narration by a voice over” as used herein can refer to words spoken by voice over to guide the listener through the story or to provide context to the story.

The term “non-holophonic sound effects” as used herein can refer to sound effects, which do not take into account natural ear spacing or head shadow of the head and ears.

The term “playback” as used herein can refer to the reproduction of an audio piece, or audio story that has a title, a beginning, a middle and an end. In embodiments, the audio story or audio recording can have chapters.

The term “visual icon” as used herein can refer to a title, an author's name, and an image that reflects characteristics of contents of the audio recording.

The term “script” as used herein can refer to a series of at least one of: sounds and words that could be sequential or at the same time, which together creates an expression that can teach, provide mental health therapy, safety training, provide a narrative, and/or strengthen cognitive skills of a listener.

The term “visual image” as used herein can refer to at least one of: a static image or a video image, including black and white or color video, photographs, graphic images, paintings, animations, cartoons, ultrasound images, infrared images, x-rays, sonograms, 3D and 2D images, computer generated simulations of 3D images or environments, and drawings.

The term “headphones”, “wearable headgear” or “ear buds” as used herein can refer to any listening device utilized to transmit sounds into a user's ear(s).

One or more illustrative embodiments incorporating the invention disclosed herein are presented below. Applicant has created a revolutionary and novel binaural device and method of use of the same.

In the following description, certain details are set forth such as specific quantities, sizes, etc. so as to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Referring to the drawings in general, it will be understood that the illustrations are for the purpose of describing particular embodiments of the disclosure and are not intended to be limiting thereto. Drawings are not necessarily to scale and arrangements of specific units in the drawings can vary.

Certain terms are used in the following description and claims to refer to particular system components. As one skilled in the art will appreciate, different persons may refer to a component by different names. This document does not intend to distinguish between components that differ in name but not function. The drawing figures are not necessarily to scale. Certain features of the invention may be shown exaggerated in scale or in somewhat schematic form, and some details of conventional elements may not be shown, all in the interest of clarity and conciseness.

Although several preferred embodiments of the present invention have been described in detail herein, the invention is not limited hereto. It will be appreciated by those having ordinary skill in the art that various modifications can be made without materially departing from the novel and advantageous teachings of the invention. Accordingly, the embodiments disclosed herein are by way of example. It is to be understood that the scope of the invention is not to be limited thereby.

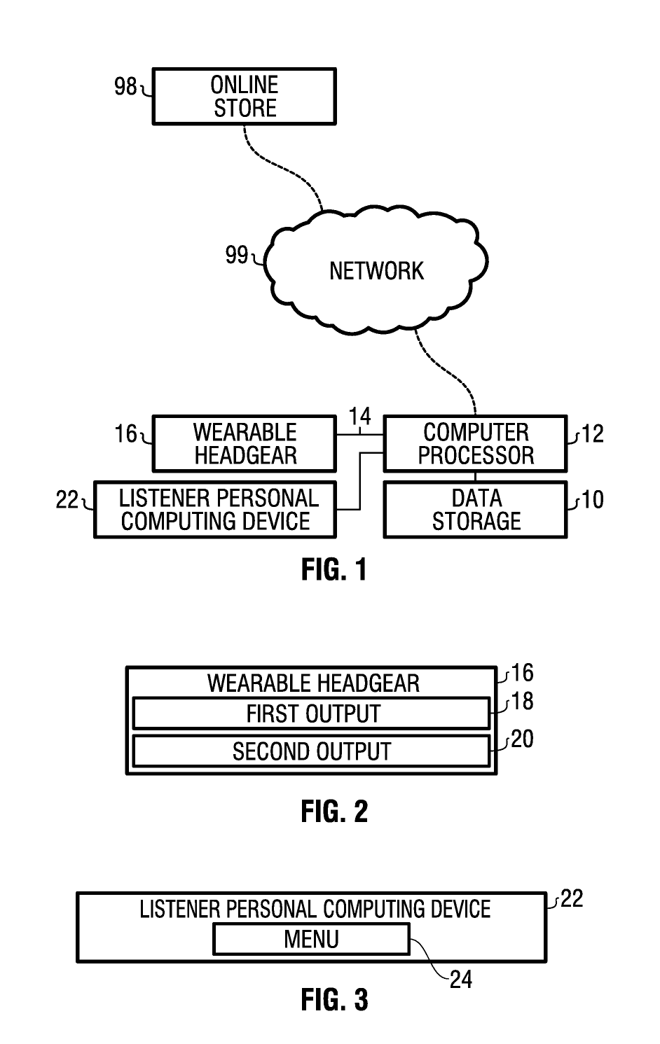

In some embodiments, FIG. 1-FIG. 3 show the system for playback of an audio recording having a binaural audio recording. In some embodiments, the system can have a computer processor 12 connected to the data storage 10. In some embodiments, data storage 10 is software. In some embodiments, the computer processor 12 can be configured to receive and playback an audio recording as an audio output signal 14. The computer processor can be, but is not limited to, a smart phone, Bluetooth, mobile computing device, laptop, tablet, personal computer, Bluetooth transmission device, or similar computing device with memory and bidirectional communication.

In some embodiments, the system can include a wearable headgear 16 in communication with the computer processor 12. The wearable headgear 16 can be configured to receive and play the audio output signal 14 through a first output 18 and a second output 20. In some embodiments, the wearable headgear 16 can include headphones, earbuds, blue tooth accessories, or any device having an audio transmitter with a direct connection to the listener's ears.

In some embodiments, the system can include a listener personal computing device 22 connected to the computer processor 12 to display a menu of audio recordings 24 in the data storage. In some embodiments of the inventive device, the listener personal computing device 22 can be a smart phone, mobile computing device, Bluetooth transmission device, headphones, laptop, ear buds, tablet, personal computer, joystick with connected display, wearable computer, eyewear, or similar device, or similar computing device, or software, with memory and bidirectional communication.

In some embodiments, the computer processor 12 can be connected to a network 99, which can communicate bi-directionally with an online store 98. In several embodiments, online store 98 can include data storage, hardware, software or cloud storage. The computer processor 12 via the network 99 can be used to browse and to download or stream audio recordings from an online store 98. In some embodiments, the network 99 can be a cellular network, a global communication network, a satellite network, a local area network, a wide area network, a drone, Bluetooth transmission or combinations thereof.

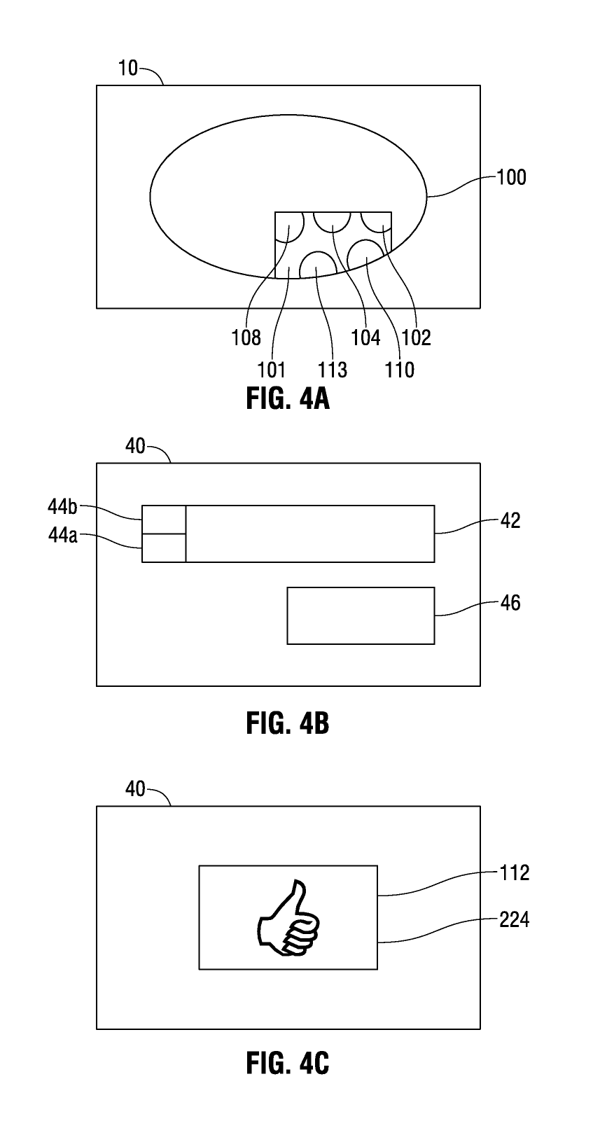

FIGS. 4A-4B shows a detail of data storage 10 with audio recording 100, as utilized in some embodiments of the present invention. In some embodiments, the data storage 10 can be a non-transitory computer readable medium that contains: at least one audio recording or a plurality of audio recordings. Each audio recording 100 can have a binaural audio recording 101. Each binaural audio recording 101 can have: a plurality of holophonic sound effects 102, a plurality of non-holophonic sound effects 104, dialog 108, a narration by a voice over 110, and optionally music 113.

In several embodiments, FIG. 4B, the binaural audio recording 40 can have a script 42, which can be a musical score, a script for a play, a sequence of machine sounds, or a series of therapy sounds. In several embodiments, the script 42 can contain a first individual audio source 44a and a second individual audio source 44b. First individual audio source 44a can be a first character, and second individual audio source 44b can be a second character, from a play. In some embodiments, the script, if it is not text, but a music score, can have an oboe part as the first individual audio source and a flute part as the second individual audio source. In some embodiments, the script is not limited to two individual audio sources. One hundred or more individual audio sources can be included in any one script simultaneously.

In some embodiments, as shown in FIGS. 4B and 4C, each audio recording 40 in addition to the binaural audio recording can have a cover icon 112 presenting (i) a title of the audio recording, (ii) a visual image, (iii) a binaural audio recording, and (iv) a URL. In some embodiments, the audio recording 40 can have non-script content, such as supplemental information 46. For example, supplemental information 46 can be a conductor narration about a piece of music or equipment specifications on machines making the machine noises when machine noises form the script.

In some embodiments, a cover icon 112 can present a title of the audio recording connected to a visual image button 224 and therein a visual image. In some embodiments, the cover icon can provide a URL that enables a listener to quickly and easily link to the audio recording related to the cover icon in the data storage. Each audio recording can also have a menu listing names and/or images of principal characters.

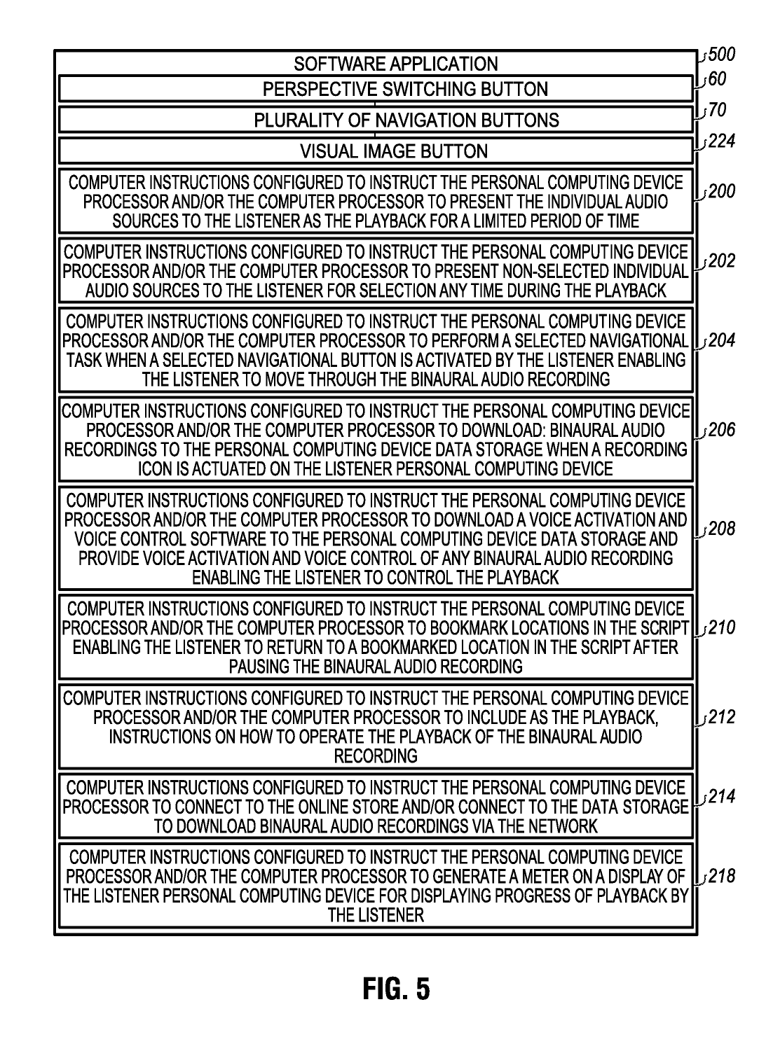

As shown in FIG. 5, in several embodiments, the data storage can contain computer instructions 200 for instructing the computer processor to present a series of audio recording choices to a listener and enable the listener to select one of the audio recordings from the menu of audio recordings each having a binaural audio recording. Also shown is software application 500 which contains data content and perspective switching button 60, which allows a user to switch perspectives in an audio recording 40 to hear different audio portions and relative audio locations. Present also are the navigation buttons 70 which allow a user to flip through different audio recordings 40.

In several embodiments, the data storage can contain computer instructions 202 for instructing the computer processor to present a menu of principal characters for the audio recording onto a listener personal computing device 22. In several embodiments, the listener can select an audio recording and a principal character by clicking on presented icons representing the audio recording and the principal character.

In embodiments, the listener personal computing device 22 (FIG. 1) can have a microphone for detecting audio commands from a listener and triggering at least one of: the recording icon, the binaural audio recording, the perspective switching button, the plurality of navigation buttons, and the visual image button enabling a listener to control the playback. In several embodiments, the data storage can contain computer instructions 204 for instructing the computer processor to playback an audio recording having a binaural audio recording from the selected principal character's perspective.

FIG. 5, in several embodiments, depict the software be application 500, which can be in the data storage or the listener personal computing device data storage 10, which needs to include hardware and software. The software application 500 can have a perspective switching button 60, and a plurality of navigation buttons 70. Additionally, the visual image button 224 can be part of the software application.

In several embodiments, computer instructions are configured to instruct the personal computing device 22 processor and/or computer processor to present non-selected individual audio sources to the listener for selection at any time during feedback 202. In several embodiments computer instruction is configured to instruct personal computing device 22 and/or the computer processor to performing selected navigational task when a selected navigational button is activated by the listener enabling the listener to move though the binaural audio recording 204. In several embodiments computer instructions are configured to instruct the personal computing device 22 processor and/or the computer processor to download or stream: binaural audio recordings to the personal computing device data storage when a recording icon is actuated on the listener personal computing device 206.

In some embodiments, computer instructions are configured to instruct the personal computing device 22 processor and/or computer processor to download or stream a voice activation and voice control software to the personal computing device 22 data storage and provide voice activation and voice control of any binaural audio recording enabling the listener to control the playback 208. In some embodiments, computer instructions are configured to instruct the personal computing device 22 processor and/or computer to processor to bookmark locations on an audio recording enabling the listener to return to a bookmarked location in the script after pausing the binaural recording 210.

In some embodiments, computer instructions are configured to instruct the personal computing device 22 processor and/or computer processor to include as the playback instruction on how to operate the playback of the binaural audio recording 212. In some embodiments, the computer instructions are configured to instruct the personal computing device 22 processor to connect to the online store and/or connect to the data storage to download or stream binaural audio recordings via a network 214. Computer instructions are configured to instruct the personal computing device 22 processor and/or computer processor to generate a meter on a display of the listener personal computing device for displaying progress of playback by the listener.

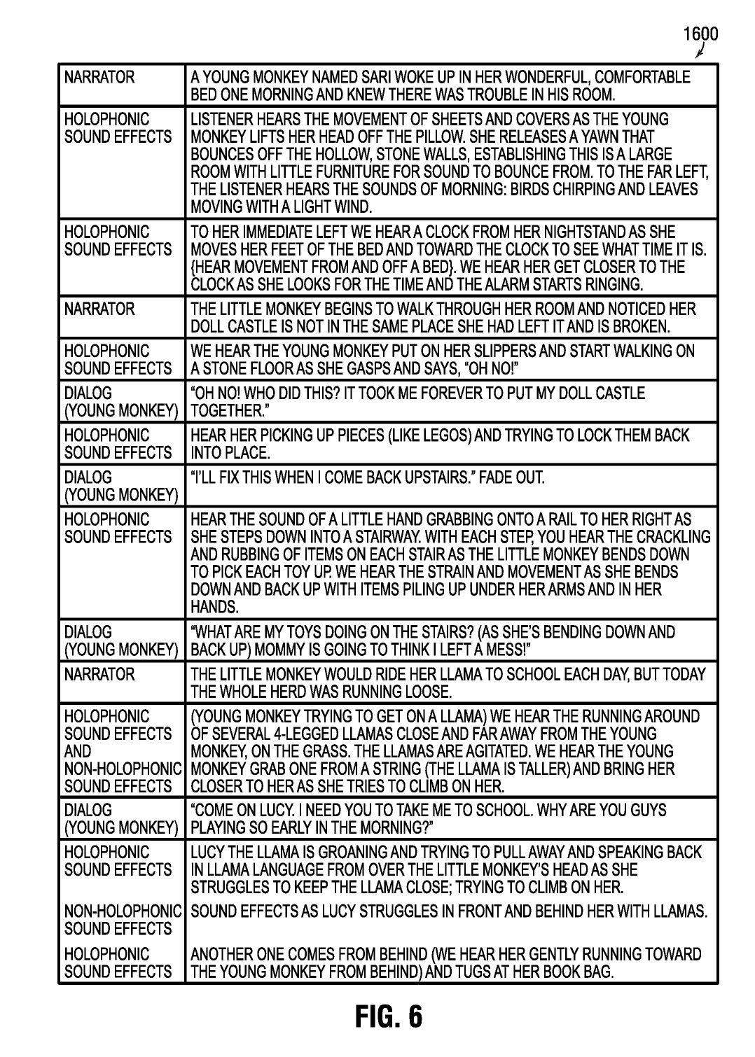

In several embodiments, FIG. 6 is a chart 600 of a binaural audio recording titled “The Monkey's Perspective”. The chart shows words from a narrator, holophonic sound effects, such as movement of sheets and covers, a clock from the nightstand, walking on a stone floor, cracking and rubbing of items on each stair; dialog from the young monkey; non-holophonic sound effects, such as picking up pieces and trying to lock them in place; and a combination of holophonic sound effects with non-holophonic sound effects, such as the young monkey trying to get onto a llama and Lucy the llama groaning and struggling with other llamas.

By another example, a system is providing a playback of a binaural audio recording of Romeo and Juliet for Maria to hear. In this embodiment, Maria downloads an app from a specific online app store via the internet to her smart phone. In this embodiment, the software app includes computer instructions that instruct Maria's smart phone to download or stream binaural audio recordings from a data storage, such as an online store or an administrative data storage connected via a network. In several embodiments, the computer instructions enable Maria to simply click on a recording icon presented on her listener personal computing device 22 through a menu. In some embodiments, the computer instructions enable Maria to control playback of a selected binaural recording using navigation buttons and perspective switching buttons presented on the personal computing device 22.

In some embodiments, the software app provides navigational buttons and other buttons to select the Romeo and Juliet binaural audio recording and control the playback into her earbuds. In some embodiments, the inventive device, stores a plurality of binaural audio recordings in the data storage of the online store. Each binaural audio recording has a recording icon connected to a script of a binaural audio recording.

In several embodiments, for Romeo and Juliet, the recording icon is a picture of a man and a woman in 13th century clothing. Maria picks the picture of the man and woman and downloads or streams the binaural audio recording to her smart phone. In several embodiments, on her smart phone, Maria sees a first individual audio source button titled “Juliet” and a second individual audio source button titled “Romeo” and a third individual audio source button titled “Spectator”. Each individual audio source button activates an audio source for playback for as long as that individual has a role in the playback being presented on the binaural audio recording. For example, in this case, Romeo's playback would end before the actual ending of the play.

In several embodiments, Maria inserts a pair of earbuds, which can be wearable headgear into her ears to receive the playback of Romeo and Juliet. The earbuds provide a first audio output into her left ear that is different from the second audio output into her right ear. Once Maria picks the Juliet button, the binaural audio recording starts the playback. In this example, instructions on how to start the playback of the binaural recording, including instructions on how to click a navigation button, and close eyes can be provided.

In several embodiments, while Maria listens to Juliet telling the story from her context, (on a second floor) from her perspective using her lines, a perspective switching button appears on the display of the smart phone. Once that perspective switching button is activated, non-selected individual audio sources of Romeo or Spectator can be presented to Maria for selection any time during the playback 10 from Juliet's individual audio source. In several embodiments, continuously during the playback, a plurality of navigation buttons can be displayed enabling Maria to move through the binaural audio recording. At lunchtime, Maria selects the “pause button” to stop the binaural audio recording and mark a location in the script enabling Maria to return to the playback marked location in the script after lunch. In some embodiments, after lunch, Maria returns to the playback and decides to select “Spectator” and hears the remainder of the story from the perspective of the “Spectator.”

In another example embodiment of one aspect of the present invention. Jose who is blind, decides to listen to a binaural audio recording. Jose wants to hear a concert from various instrument perspectives because he is an aspiring conductor. In this embodiment, Jose puts on his wearable headgear, headphones, with a processor and data storage and Bluetooth connectivity to receive playback and provide a first audio output and a second audio output to his ears. In this embodiment, his listener personal computing device, Jose uses the processor and data storage mounted in the headphones to control playback to the wearable headgear.

In several embodiments, Jose connects to a computer processor via the Internet to download or stream computer instructions into the processor in his headphones that enable him to download or stream binaural audio recordings by using voice activation and voice control software. The voice activation software enables Jose to solely control playback with the wearable device. In many embodiments, the wearable device is compatible with any downloadable or streaming playback.

In several embodiments, Jose's headphones include a microphone connected to the personal computing device processor for detecting audio commands from Jose and triggering at least one of the recording icons in data storage connected to an online app store. In this case, Jose simply says “Play the Conga Line” and the voice recognition software in the data storage connects Jose to the correct binaural audio recording. In several embodiments, the voice recognition software can enable Jose to control the playback of the binaural audio recording including selection of a perspective by vocally engaging the perspective switching button and vocally engaging one or more of the plurality of navigation buttons enabling a listener to control the playback feature.

In several embodiments, Jose's personal computing device connects to a computer processor via the internet that has a data storage with many binaural recordings to select from and download or stream and enjoy. Jose can decide to select the trumpet from “Play Conga Line” as his first individual audio source. About 10 minutes into the playback, Jose decides he wants to hear the song from the perspective of the drums. Jose speaks “Activate perspective switching.” The software speaks the non-selected individual audio sources which include “drums” into a speaker of the listener personal comping device that Jose hears. Jose then says the words “drums”. The binaural audio recording then continues playback from the drum player's perspective.

In several embodiments, the listener's personal computing device 22 can be affixed to the wearable headgear. In some embodiments, the script can be a music score and the individual audio sources of the script can be instruments presenting a portion of the music score or an individual audio source can be from the perspective of the audience viewing the presentation of the music score. In some embodiments, the script can be a series of machine sounds in a manufacturing or production environment.

In some embodiments, the script can be a series of neurological therapy sounds for meditation, motivation, hypnosis therapy, relaxation, and improved concentration. In some embodiments, the script can be a play and the individual audio sources can be characters in the play or the audience viewing the play. In some embodiments, the script can be sequentially ordered sound effects to diagnose hearing impairment or diagnose hearing improvement. In some embodiments, the listener's personal computing device can be a smart phone, mobile computing device, laptop, tablet, personal computer, headphones, earbuds, or similar computing device with memory.

In some embodiments, the wearable headgear can be headphones, earbuds, Bluetooth peripherals, or any device having an audio transmitter with a direct connection to each of the listener's ears.

In some embodiments, the computer processor and data storage can be at least one of: a smart phone, mobile computing device, laptop, tablet, personal computer, headphones, ear buds, or similar computing device with memory. In some embodiments, the network can be a cellular network, a global communication network, a drone, a satellite network, a local area network, a wide area network or combinations thereof.

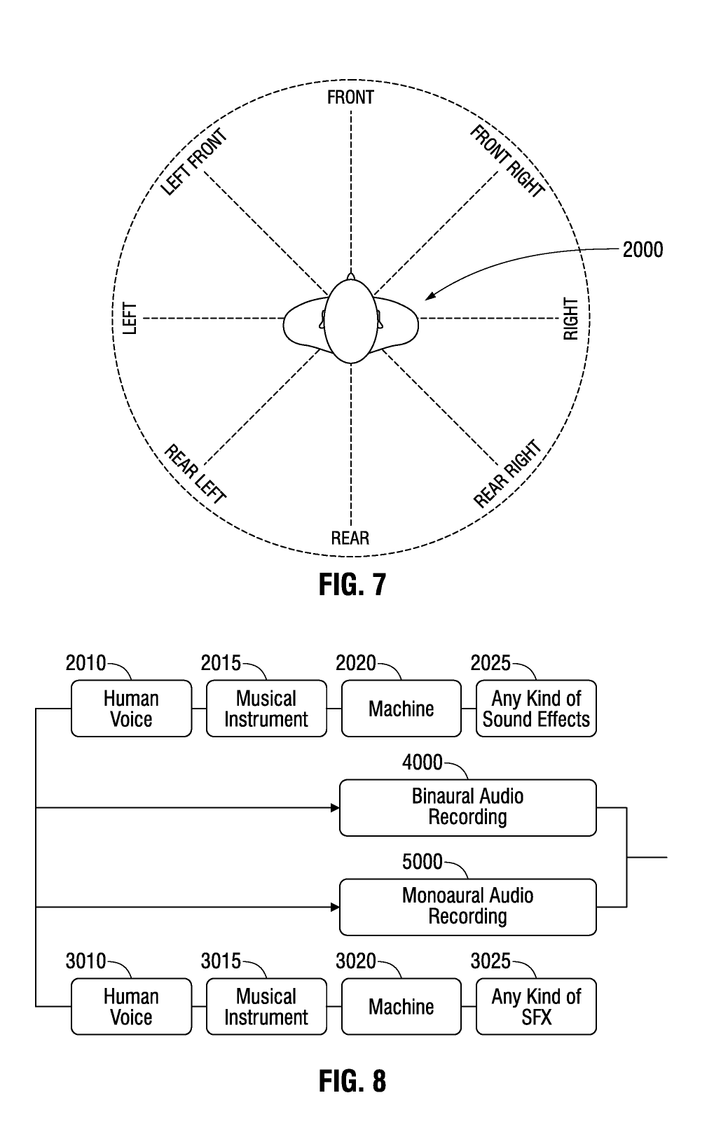

FIG. 7 illustrates one embodiment of the present invention in which a user 2000 is shown graphically in relationship to a variety of potential sound input locations. The ability to perceive sound direction works through a process known as binaural hearing, which essentially means “hearing with two ears”. The key element to binaural hearing is the location of the ears and altered by the elements and details that affect how the sound is picked up: the size and shape of the head, its surroundings and their composition, distance; among others. Additionally, because the ears are on opposite sides of the head, the sounds heard by either ear will vary in timing, volume, and frequency balance. These differences are the clues the brain uses to decode a sound's location. If we want to create audio illusions, we must understand and mimic how the ears interpret information to trick them and have them work with the brain toward the desired objective.

Generally, the brain interprets each audio stimuli as follows: Variations in Timing—When a sound comes from the left, the travel distance to the left ear is-slightly shorter than the travel distance to the right ear. So, the left ear hears it milliseconds before the right. That's the brain's first signal that the sound might be coming from the left. Variations in Volume-Sound gets softer as it moves further away. Sound also gets softer when there are objects blocking it. If the unidentified sound is coming from the left . . . . It will also sound a tiny bit softer to the right ear since it's further away. And it will sound softer still, since the head is blocking it. Variations in Frequency—When a sound comes from the left, the head blocks a portion of it from reaching the right ear. However, it doesn't block all frequencies equally. High frequencies have less energy, are more easily absorbed by obstructions than low frequencies. So, with the unidentified sound, the right ear will get more of the low end, and less of the high-end frequencies.

When all timing, volume and frequency combine, the brain identifies what it hears. Because in nature, these 3 elements always line up. If the sound is directly in front of a person: The timing would be the same in each ear. The volume would be the same. The frequency would be the same. And the same would also be true if the sound were directly behind a person.

Distance: When the brain judges the distance of a sound, it relies less on binaural hearing, and more on the following considerations: the frequency response, the amount of reverb, and the amount of pre-delay. Amount of Reverb—The farther the sound, the more reverb it has. That “reverby” character of far-off sounds exists because almost none of the sound reaches a person directly. Instead, almost all of it reflects off multiple surfaces before ever reaching the ears. That is the brain's first notice that a sound is far away. Pre delay- Pre-delay is the time gap between the first arrival of direct sound, and the first arrival of reflected sound. In an environment with lots of reflective surfaces, sounds heard from up close may still have a lot of reverb, but the time gap between the directed sound and reverb will be large. Far-off sounds have a shorter pre-delay time, because both sounds must travel a great distance to reach the person. Frequency Response—When a sound travels far to reach a person, much of its high frequency detail dissipates along the way. High frequencies carry less energy than low ones, so they are more easily absorbed by objects in the environment. Over long distances, obstructions such as land masses, buildings, and even air in the atmosphere all contribute to squash those high frequencies. When all these sound elements align, the ears and the brain connect and process all the information to identify what is being heard.

Binaural recordings utilize frequency, pre-delay, reverb, volume and timing, to generate different audio effects than mono recordings.

FIG. 8 illustrates one embodiment of content creation of binaural content. The perspective spatial 3D audio effect is created by recording (through single/stereo/microphones, or 3D microphones with ear-like cavities or holophones; among others) audio stimuli (voices, sound effects (“SFX”), instruments—anything that generates sound) directly into suitable formats for multi-channel or binaural reproduction. Or the spatial effect can be created artificially in any two/multi-channel audio signal. Monoaural audio recording 5000 can be created with human voice 3010, musical instrument 3015, machine 3020, and any kind of SFX 3025. Binaural audio recording 4000 can be created with human voice 2010, musical instrument 2015, machine 2020, and any kind of SFX 2025.

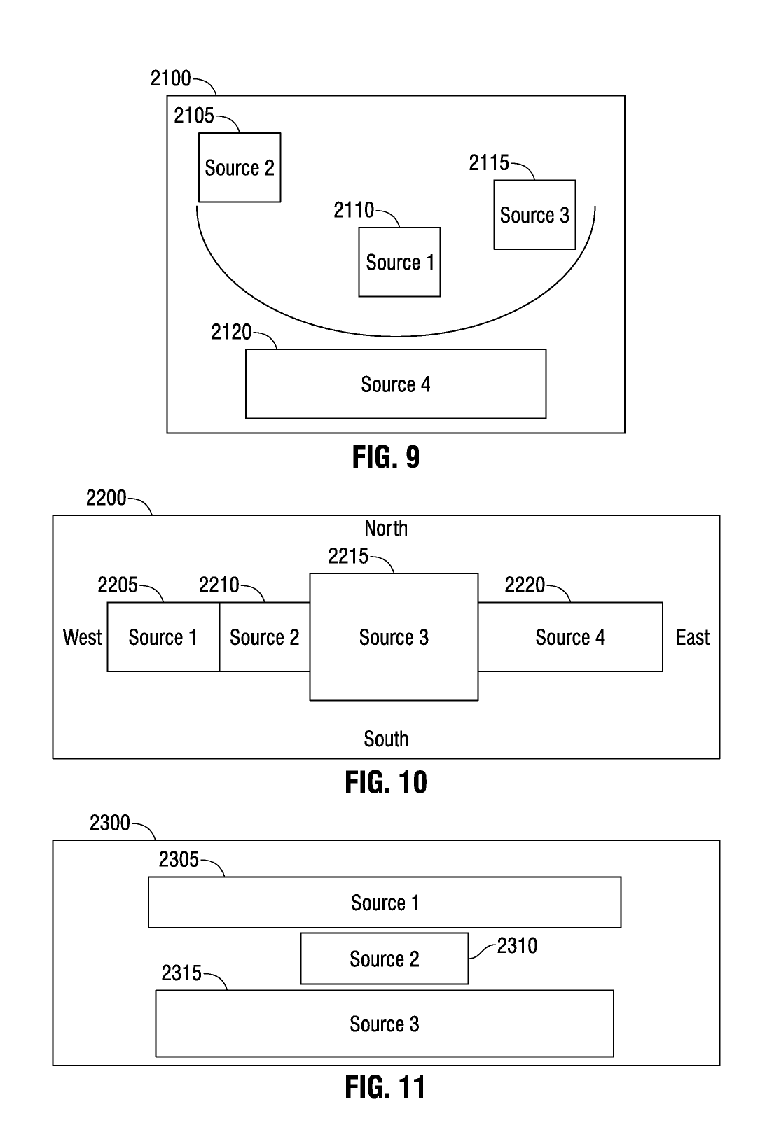

FIG. 9 illustrates one embodiment of the present invention as an example of music score/concert. As shown, a binaural audio recording of the music band The Dudes, where the script is their hit song “Today”, and the sources for playing back the script are Jeff (vocals) 2110, Roger (drums) 2105, George (guitar) 2115 and Audience 2120. The listeners, through their personal computing device (smartphone screen), are able to select which script they want to listen to and from which source's perspective Jeff, Roger, George, or Audience.

In several embodiments, if the listener selects source 1, the playback of the binaural audio recording will be from Jeffs perspective 2110, which means that the listener will “become” the lead vocal in the script, with source (drums) 2105 sound coming from back-right, source (guitar) 2115 sounds coming from side-left, and source (audience) 2120 sound coming from far-front.

FIG. 10 illustrated one embodiment of the present invention using “Machinery.” In this embodiment, a binaural audio recording of the plastic bottle-building machine at the soda bottling facility, where the script 2220 are the sounds stemming from the process (plastic injection 2205, bottle contouring 2210, plastic freezing 2215, end product exit 2220), and the sources are the different parts of the machine (injectors, molds, freeze hose, conveyor belt).

In several embodiments, if the listeners are maintenance workers in training who, through their personal computing device (smartphone screen), are able to select from which source's perspective (injectors 2205, molds 2210, freeze hose 2215, conveyor belt 2220) they want to listen to the script (bottle-building process) 2200.

In several embodiments, if a listener selects to stand in front of source on the north side, the playback of the binaural audio recording will be from that perspective, giving the listener the ability to more closely hear source 2205 and 2210 sounds, while source 2215 and 2220 sounds will be heard far away. If the listener selects to stand in front of the same source 2205, but on the south side, different sounds in different positions will be heard according to perspective. In several embodiments of the present invention, perspective will enable listener to understand normal sounds vs. abnormal sounds, and a training manual associated to abnormal sounds will allow the maintenance worker to detect and solve malfunctioning machinery.

FIG. 11 illustrates another embodiment of the present invention as used in conjunction with a sporting event 2300. As shown, a binaural audio recording of a racing pit area, where the script is the behind-the-scenes of team Jimmy pit area at the 2016 West Grand Prix, and the sources for playing back the script are Sebastian Victor (driver 1) 2305, Team Jimmy (pit crew) 2310 and the Fan Seats 2315. The listeners, through their personal computing device (smartphone screen), are able to select from which source's perspective (Sebastian, Pit Crew, or Fan Seats). As shown, if listener selects source 2305, the playback of the binaural audio recording will be from one of the driver's perspectives, which means that the listener will “become” the driver in the script, and will be able to listen to what happens inside the cockpit of the Formula 1 car, along with the radio communication the driver is receiving, and the sounds associated with the pit stop. Sounds from source 2310 (pit crew) will be felt all around as the tires are changed and the car is refueled, while sounds from source 2315 will be heard to far-left from people cheering.

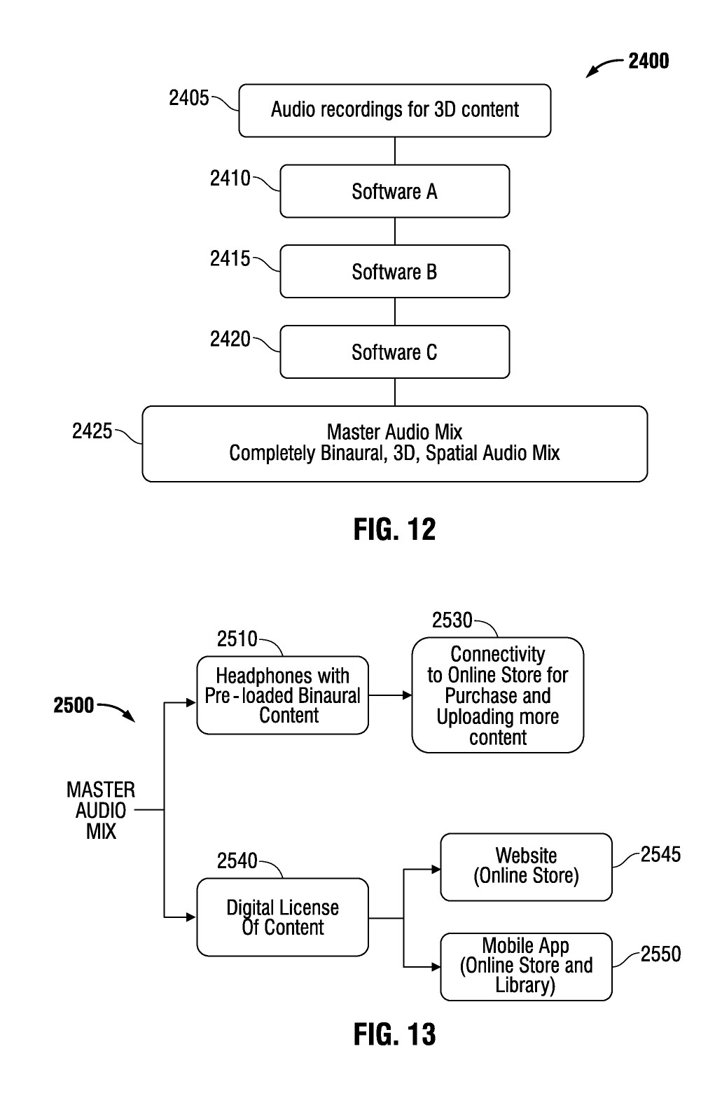

FIG. 12 illustrates another embodiment of the present invention as an audio recording is edited, optimized, mixed and mastered with the goal to generate spatial perspective 2400. As shown audio recordings for 3D content 2405 are edited, optimized, mixed and mastered using combinations of software A 2410, software B 2415, and software C 2420. The generated result is preferably master audio mix 2425 which is completely binaural, 3D and a spatial audio mix.

FIG. 13 illustrates several embodiments of the present invention as the master audio mix 2500 can be present in headphones, or ear buds, with pre-loaded binaural content 2510 which can then use a direct, wireless, or Bluetooth connectivity to an online store to purchase and upload more content 2530. In alternative embodiments, digital download or streaming of content 2540 is possible from a website 2545 or from a mobile application 2550.

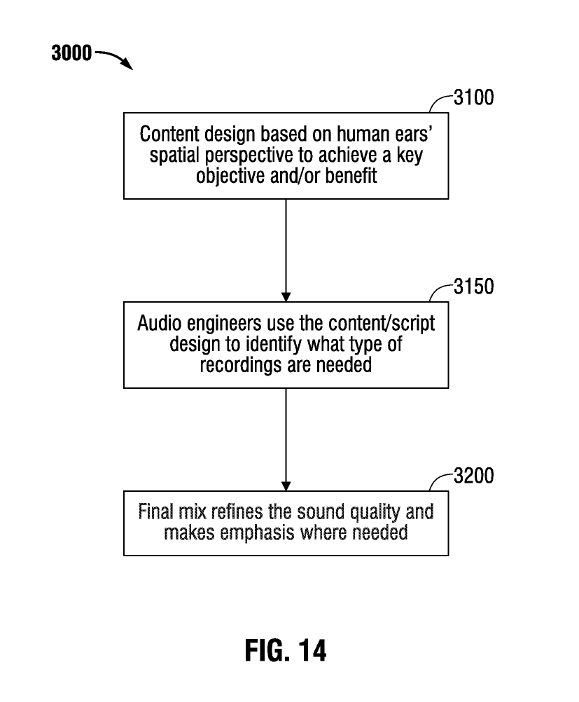

FIG. 14 illustrates one embodiment of the steps to create binaural content and materials. As shown the binaural content and material creation 3000 is created in the following manner in one embodiment of the present invention:

In several embodiments, the first step is focused on content design based on human ears' spatial perspective to achieve a key objective and/or benefit 3100. One objective of the script to create a map of how an individual will experience different sound sources from different perspectives. In the script or sound map, the audio experience is outlined, scripted and/or designed for it to make sense from a spatial perspective purely through the ears. So, sounds coming from the: left, right, front, back, top, bottom, and so forth, have to make sense to the human brain to convey an objective.

For example, if the objective of the audio experience is to help a blind person understand distance and location of him/herself vs. surroundings, the audio source is recorded with that spatial perspective (distance) and location vs. the human being. If the objective is to experience a concern from the violins' section vs. the trumpets section, the music is recording from that perspective and location, however, if the listener wants to switch from violins to the conductor, this enables the listener to do so. The audio experience can also feature movement from a story character. So, in Peter Pan, Captain Hook can move from left to right during a sword fight and the listener can experience it from Peter Pan's perspective. The listener can also switch to experience the story from Tinker Bell's perspective, and experience the same story by flying above what's happening, or by becoming part of the plot and getting trapped by Captain Hook. The same concept applies to therapy. Sounds, the intensity of sounds or messages and/or repetition from left vs. right audio consumption, will have different health implications and effects.

In several embodiments, in the next step 3150, audio engineers use the content/script design to identify what type of recordings are needed. This will include but is not limited to: 3D audio recording, mono audio recordings, binaural beats, Foley and regular, and/or prerecorded sound effects. All the audio elements need to be recorded and then edited to achieve the audio experience objective.

In several embodiments, the final step 3200 commences, once the final edit is complete, the final mix 3200 refines the sound quality and makes emphasis where needed. This might include increasing or lowering the volume at key points within the recording; among many other final enhancements; depending on the objective. The final output is an audio experience that tricks the human brain into thinking it is experiencing something as if in the middle of the audio stimuli; when the person is physically absent.

In embodiments, instead of using surround sound techniques, which use multiple speakers placed in different locations, binaural audio recordings can establish direction and distance between the listener and different audio elements. For example, the listener can determine the location of a bird chirping from a tree in front of him or a cricket chirping from the grass behind him.

In some embodiments, once the binaural audio recording starts the playback of play, individuals use their imagination to be transported into an era or place wherein even though the listener is not physically at the play venue, the person feels they can be part of the audience or part of the cast of the play.

Some embodiments can create a spatial reality all around the listener. Also, music can be a vital component of the binaural audio recording. In some embodiments, when the script is a music score, a listener can select any one seat in a theater to hear the music from that acoustic location. In some embodiments, when the script is music, the listener can be the third seat trumpet player and simultaneously hear the entire score of the orchestral piece from the seat of the third trumpet player, teaching the third trumpet player how to listen and react to other instruments/players of the orchestra.

While these embodiments have been described with emphasis on the embodiments, it should be understood that within the scope of the appended claims, the embodiments might be practiced other than as specifically described herein.

I claim:

1. A system for playback of an audio recording with a binaural audio recording, comprising:
an audio recording comprising:
a binaural audio recording;
a menu list with several features;
a menu selection button to select features from the menu listing;
a data storage configured to store at least one audio recording having the binaural audio recording and to present a series of audio recording choices to a listener, the binaural audio recording comprising a member, or combination of members, from the group consisting of: holophonic sound effects, a dialog, music, narration, machinery, environmental sounds, and/or script;
a computing system comprising:
a computer processor connected to the data storage, the computer processor configured to receive and playback the audio recording as an audio output signal;
wearable headgear in communication with the computer processor configured to receive and play the audio output signal through a first output and a second output;
a listener's personal computing device connected to the computer processor;
said data storage further comprising:
computer instructions for instructing the computer processor to present the series of audio recording choices to the listener and enable the listener to select one of the audio recordings from a series of audio recording choices using the listener personal computing device, each audio recording having the binaural audio recording;
a perspective switching button viewable, physically available, or audio activated, on the display and connected to computer instructions configured to instruct the personal computing device processor and/or computer processor to present non-selected individual audio sources to the listener for selection at any time during the playback; and
a plurality of navigation buttons viewable, physically available, or audio activated, on the display and connected to computer instructions to instruct the personal computing device processor and/or computer processor to perform a selected navigational task when a selected navigational button is activated by the listener enabling the listener to move through the binaural audio recording.

2. The system of claim 1, wherein the data storage comprises computer instructions configured to instruct the personal computing device processor and/or computer processor to download or stream a voice activation and voice control to the personal computing device data storage and provide voice control of any binaural audio recording enabling a listener to control the playback, and wherein the listener personal computing device comprises a microphone for detecting audio commands from a listener and triggering at least one of: the recording icon, the binaural audio recording, the perspective switching button and the plurality of navigation buttons.

3. The system of claim 1, wherein the listener's personal computing device is from the group consisting of: a physical or software-based data storage device smart phone, mobile computing device, laptop, tablet, personal computer, wearable headgear, Bluetooth device or similar computing device with memory.

4. The system of claim 1, wherein the wearable headgear is from the group consisting of: headphones, earbuds, blue tooth accessories, or any device having an audio transmitter with a direct connection to the listener's ears.

5. The system of claim 1, the computer processor and data storage comprise at least one of from the group consisting of: data storage service, data storage hardware, USB, memory cards, a smart phone, mobile computing device, laptop, tablet, personal computer, wearable headgear, Bluetooth device, or computing device with memory.

6. The system of claim 1, comprising computer instructions for instructing the computer processor to connect the computer processor to a network to download or stream additional audio recordings from an online store.

7. The system of claim 6, wherein the network is from the group consisting of: a cellular network, a global communication network, a drone, a satellite network, a local area network, blue tooth transmission, a wide area network or combinations thereof.

8. The system of claim 1, wherein the binaural audio recording allows the listener to define sound quality metrics for size, intensity, position, distance, dynamics, tempo, type of sound.

9. The system of claim 1, the binaural audio recording further comprising at least one of: a non-holophonic sound effect and/or music.

10. The system of claim 1, wherein the script is a music score and the individual audio source is an instrument presenting a portion of the music score or the individual audio source is an audience viewing the presentation of the music score.

11. The system of claim 1, wherein the script is a series of machine sounds in a manufacturing or production environment.

12. The system of claim 1, wherein the script is a series of neurological therapy sounds for at least one of: meditation, motivation, hypnosis therapy, relaxation, and improved concentration.

13. The system of claim 1, wherein the script is a play and the individual audio source is a character in the play or the audience viewing the play.

14. The system of claim 1, wherein the network is from the group consisting of: a cellular network, a global communication network, a drone, a satellite network, a local area network, Bluetooth transmission, a wide area network, or combinations thereof.

15. The system of claim 1, wherein the personal computing device data storage comprises voice recognition software.

16. The system of claim 1, wherein the binaural audio recording comprises a visual image, a physical button, or is audio activated for an individual audio source, the visual image, a physical button, or and audio activated button linked to at least one visual image, corresponding to a portion of the playback.

17. A system for the creation of the binaural materials comprising the following steps:
designing content based on human ears' spatial perspective to achieve a key objective and/or benefit;
utilizing the content/script design to identify what type of recordings are needed for binaural effects in an audio mix; and
refining the audio mix to trick the human brain into thinking it is experiencing something as if in the middle of the audio stimuli; when the person is physically absent from said stimuli.

18. A system for playback of an audio recording with a binaural audio recording, comprising:
an audio recording comprising:
a binaural audio recording;
a menu selection button to select features from a menu listing;
a data storage configured to store at least one audio recording having the binaural audio recording and to present a series of audio recording choices to a listener;
a computing system comprising:
a computer processor connected to the data storage, the computer processor configured to receive and playback the audio recording as an audio output signal;
transmission gear in communication with the computer processor configured to receive and play the audio output signal through a first output and a second output; and
a listener's personal computing device connected to the computer processor.
